(19) United States
(12) Patent Application Publication
(10) Pub. No.: US 2009/0017129 A1
(43) Pub. Date: Jan. 15, 2009

(54) Dead Sea minerals enriched anti-inflammatory pharmaceutical composition for treatment of cutaneous dryness, itching, peeling and tightness, especially in hemodialysis patients and preparation and treatment methods thereof

(76) Inventors: Ze'ev MA'OR, Kalia (IL); Michael Royz, Jerusalem (IL); Sharon Makov, Jerusalem (IL); Tamar Tzioni, Jerusalem (IL); Miriam Oron Mingelgrin, Jerusalem (IL)

Correspondence Address: Fleit Gibbons Gutman Bongini & Bianco PL, 21355 EAST DIXIE HIGHWAY, SUITE 115, MIAMI, FL, 33180, US

(21) Appl. No.: 12/171,076

(22) Filed: Jul. 10, 2008

Related U.S. Application Data
(60) Provisional application No. 60/929,840, filed on Jul. 13, 2007.

(51) Int. Cl. A61K 33/00; A61P 29/00; A61P 17/00

(52) U.S. Cl. 424/600

(57) ABSTRACT

This invention relates to Dead Sea anti-inflammatory medicaments or cosmetics for treatment of anti-inflammatory conditions; for example, cutaneous dryness, itching, peeling, and tightness, especially in hemodialysis patients. Methods for the preparation of and methods of treatment using these medicaments or cosmetics are also provided.

### FIELD OF THE INVENTION

The present invention generally pertains to Dead Sea anti-inflammatory medicaments or cosmetics for treatment of anti-inflammatory conditions, especially e.g., cutaneous dryness, itching, peeling and tightness, especially in hemodialysis patients

### BACKGROUND OF THE INVENTION

Cutaneous manifestations associated with uremia including pruritus have been estimated to be prevalent in approximately 80% of hemodialysis patients (See Chen Y C, Chiu W T, Wu M S., Therapeutic effect of topical gamma-linolenic acid on refractory uremic pruritus. Am J Kidney Dis. 2006; 48:69-76). Pruritus has been observed to be inversely associated with dialysis efficacy (Kt/v) (See Dyachenko P, Shustak A, Rozenman D., Hemodialysis-related pruritus and associated cutaneous manifestations. Int J Dermatol. 2006; 45:664-7). Male gender, blood urea nitrogen, beta-2-microglobulin, hypercalcemia, and hyperphosphatemia have been observed to be independent risk factors for the development of severe pruritus, while low serum calcium and PTH levels have been associated with reduced risk (See Narita I, Alchi B, Omori K, Sato F, Ajiro J, Saga D, Kondo D, Skatsume M, Maruyama S, Kazama J J, Akazawa K, Gejyo F. Etiology and prognostic significance of severe uremic pruritus in chronic hemodialysis patients. Kidney Int. 2006; 69:1626-32). Because lower serum albumin and higher serum ferritin levels have been reported in pruritic compared to nonpruritic uremic patients, an association between pruritus, malnutrition and inflammation has been proposed (Lugon J R. Uremic pruritus: a review. Hemodial Int. 2005 April; 9:180-8). More ominously, severe pruritus has been identified as an independent predictor of death even after adjusting for other clinical factors including diabetes mellitus, age, beta2MG, and albumin (Narita et al.).

The physiologic underpinnings of pruritus and other cutaneous manifestations observed in uremic patients have not been fully elucidated but could be associated with dermal mast cells. Specifically, pruritus intensity has been shown to be significantly correlated with mast cell tryptase levels, an associated not observed with other mast cell-related parameters (See: Dugas-Breit S, Schopf P, Dugas M, Schiffl H, Rueff F. and Przybilla B. Baseline serum levels of mast cell tryptase are raised in hemodialysis patients and associated with severity of pruritus. J Dtsch Dermatol Ges. 2005; 3:343-7).

Regardless of its etiology, cutaneous manifestations of uremia are debilitating for the dialysis patient and a number of therapies have been attempted to reduce patient discomfort. Orally administered therapies include the kappa opiod nalfurafine (See: Wikstrom B., Gellert R., Ladefoged S D., Danda Y., Akai M., Ide K., Ogasawara M., Kawashima Y., Ueno K., Mori A. and Ueno Y. Kappa-opioid system in uremic pruritus: multicenter, randomized, double-blind, placebo-controlled clinical studies. J Am Soc Nephrol. 2005; 16:3742-7); gabapentin (See: Manenti L, Vaglio A, Costantino E, Danisi D, Oliva B, Pini S, Prati E, Testori A. Gabapentin in the treatment of uremic itch: an index case and a pilot evaluation. J Nephrol. 2005; 18:86-91); and n-3 and n-6 polyunsaturated fatty acid supplements (See Begum R, Belury M A, Burgess J R, Peck L W. Supplementation with n-3 and n-6 polyunsaturated fatty acids: effects on lipoxygenase activity and clinical symptoms of pruritus in hemodialysis patients. J Ren Nutr. 2004; 14:233-41). The systematic use of these medications is an obvious disadvantage.

Total body irradiation with UVB phototherapy has been employed (See Lazrak S, Skali H, Benchikhi H, Lakhdar H, Fatihi M, Ramdani B, Zaid D. Phototherapy and hemodialysis pruritus. Nephrologie. 2004; 25:293-5), the down side of such a treatment is the known risk of UVB radiation to induce carcinoma, especially in a long term repeated treatments is needed

Alternative therapies as acupuncture (See: Che-Yi C, Wen C Y, Min-Tsung K, Chiu-Ching H. Acupuncture in haemodialysis patients at the Quchi (LI11) acupoint for refractory uraemic pruritus. Nephrol Dial Transplant. 2005; 20:1912-5) and homeopathic remedies (See Cavalcanti A M, Rocha L M, Carillo R Jr, Lima L U, Lugon J R. Effects of homeopathic treatment on pruritus of haemodialysis patients: a randomized placebo-controlled double-blind trial. Homeopathy. 2003; 92:177-81), were employed as well.

The closest prior art treatment to the invention presented hereupon is a topical application of gamma-linolenic acid (2.2%) body lotion was found to be more effective than placebo cream in relieving pruritus (See Chen Y C, Chiu W T, Wu M S. Therapeutic effect of topical gamma-linolenic acid on refractory uremic pruritus. Am J Kidney Dis. 2006; 48:69-76).

The advantages of treatment of skin disorders with Dead Sea mud and/or water are well documented in the literature. Recently, in patents granted to one of us in collaboration with others, U.S. Pat. No. 6,582,709 and WO 0040255 (A1) is disclosed the composition, method of preparation and use of a cream based on Dead Sea mud and Dead Sea water for medical and/or cosmetic treatment of skin that overcomes prior problems incorporated with mixing of Dead Sea mud and/or Dead Sea water in creams. The cream presented thereof was found to be efficient in the treatment of the following skin disorders and diseases: Psoriasis, Seborrehic dermatitis, Xerosis (very dry skin), Atopic dermatitis, Eczema, Diaper rush, sensitive skin, and skin burns of stage I.

At present, no widely accepted treatment for uremic pruritus exists. A new method of treatment is therefore desirable and awaited for by dermatologists, dialysis experts and patients alike. Furthermore, there is no prior-art that uses topical application with pharmaceutical composition enriched with Dead Sea minerals to treat symptoms of uremic pruritus.

### SUMMARY OF THE INVENTION

It is one object of the present invention to disclose a safe and effective Dead Sea mineral enriched anti-inflammatory pharmaceutical composition, especially useful for treatment of symptoms of uremic pruritus including cutaneous dryness, itching, peeling and tightness, especially in hemodialysis (HD) patients.

It is further an object of this invention to provide a method for preparation of said Dead Sea minerals enriched anti-inflammatory pharmaceutical composition.

It is yet further an objective of this invention to disclose a method for treatment of symptoms of skin inflammatory conditions, and especially uremic pruritus including cutaneous dryness, itching, peeling and tightness, especially in hemodialysis patients by application of said Dead Sea minerals enriched pharmaceutical composition.

The Dead Sea minerals enriched anti-inflammatory pharmaceutical composition disclosed in this invention comprises a combination that includes: (1) basic ingredients for preparation of an excipient phase such as an emulsion, gel or powder compatible for application on a skin of a human such. In a preferred embodiment the excipient phase is an emulsion of any form (e.g., normal emulsion, reverse emulsion, nano-emulsion, etc.), which comprises distilled water, oils, emulsifiers, thickeners, and preservatives; (2) commonly used active ingredients with therapeutic effects such as but not limited to herbal extracts, vitamins, anti-oxidants and amino acids (3) Dead Sea mud and possibly Dead Sea water.

The preferred method of preparation of said anti-inflammatory pharmaceutical composition is carried out by preparing an emulsion which contains all the aforementioned ingredients beside the Dead Sea mud and/or water, and mixing the latter two with the emulsion at 25° C. as described e.g., in U.S. Pat. No. 6,582,709 which is incorporated herein as a reference.

The preferred method of treatment is by topical administration of the said Dead Sea minerals enriched anti-inflammatory pharmaceutical composition to the skin suffering from a disorder; and leaving said Dead Sea minerals enriched composition on said skin until at least substantially all of said Dead Sea minerals have been absorbed by the skin and said disorder has been ameliorated. The treatment is repeated several times as long as needed preferably 1-4 times a day for 1-30 consecutive days ideally twice a day for 14 consecutive days, preferably after rinsing the skin.

### BRIEF DESCRIPTION OF THE DRAWINGS

FIG. 1 discloses the protection effect of Dermud against inflammation induced by UVB irradiation.

FIG. 2 discloses the effect of Dermud on reducing inflammatory cytokines.

### DETAILED DESCRIPTION OF THE INVENTION

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the general principles of the present invention have been defined specifically to provide (1) a pharmaceutical composition enriched with Dead Sea minerals for treatment of anti-inflammatory conditions, especially pruritus and related symptoms including cutaneous dryness, itching, peeling and tightness, especially in hemodialysis patients; (2) a method for preparation of said composition in a preferred embodiment as an emulsion; (3) a treatment method of said disorders by application of the aforementioned pharmaceutical composition.

The term ‘Dead Sea minerals’ refers in the present invention to any inorganic composition originating from the Dead Sea. Said composition is provided from the Dead Sea as is, or as at least partially dried mud, salt, salt solution, suspension, powder, milled powder, pulverized powder, powder of nano-particles or any combination thereof. The composition of the aforementioned Dead Sea minerals may vary, and typically comprises major constituents as follows: MgCl₂, 30.0-34.0%; NaCl, 8.0-18.0%; KCl, 22.0-28.0%; CaCl₂, 00.3-00.7%; SO₄⁻², 00.1-00.2%; and Br⁻, 00.2-00.4%. Dead Sea salt may further comprise minor constituents as follows: Rb, 300-400 ppm; PO₄, 50-60 ppm; Fe, 20-40 ppm; St, 100-200 ppm; BO₃, 5-10 ppm; Li, 2-4 ppm; Mn, 2-4 ppm; Al, 5-10 ppm; and Cs, 1-2 ppm. Typical clay composition may vary, yet typically comprises the following ingredients: Illite-semectite phases, 50-70%; Kaolinite, 10-20%; Illit, 10-15%; Calcite, 5-15%; Quartz, 1-5%; Chlorite is less then 5%; and Palygorskite less then 5%. Elemental chemical analysis performed by Inductive Coupled Plasma (ICP) method and wet analytical analysis methods provided a typical element content (ppm) as follows: Si, 107,500; Ca, 105,300; Cl, 40,500; Fe, 24,500; Al, 22,500; Mg, 18,600; Ti, 2,700; P, 1,300; S, 1,200; Br, 1,200; Sr, 400; Mn, 225; Ba, 200; Cr, 58; Zn, 48; Ni, 25; Li, 17; Cu, 11; Co, 7; Pb, 5; Th, 4; As, 2.5; U, 2.3; Mo, 1.5; Sn, 1; Ag, <I; Cd, 0.6; Be, 0.5; and Sb, 0.2.

Dead Sea mud (silt) as defined in the present invention is physically characterized at room temperature as a stable black paste with specific density of 1.6-1.8 g/ml. The chemical characterization of Dead Sea mud is natural sediment, mixture of solid mineral clays with interstitial solution of inorganic salts and microbiological sulfide metabolites with particle size 86-98%<0.005 mm, 2-9% 0.005-0.02 mm, 0-7%>0.02 mm, pH is from about 6.4 to about 7.6, 30-40% w/w water and less than 100 cfu/gr and non-pathogenic microbes.

The major constituents of the Dead Sea mud are:

(1) The solid phase which is 60-70% of the total weight consists of:

water soluble: Halite (20-40%); HCl soluble: carbonates (30-40%): calcite, dolomite, aragonite; non HCl-Soluble matter: silicates (30-40%): quarts, montmorollonite, feldspar, kaolonite;

(2) In the liquid phase (30-40% dissolved as ions in water):

chloride (Cl⁻) 148-190 g/l sodium (Na⁺) 22-32 g/1 magnesium (Mg²⁺) 30-40 g/l calcium (Ca²⁺) 10-15 g/l potassium (K⁺) 6-8 g/l.

Dead Sea Water, as defined in the present invention, comprises a clear colorless viscous liquid (at room temperature) with a specific density of 1.2-1.36 g/ml, pH (at room temperature) is from about 4.5 to about 6.5, and preferably is 5.5. Microorganism status is less than 100 cfu/gr and non pathogenic microbes.

The major constituents of the Dead Sea water are: Calcium (Ca⁻²) 36,000-40,000 mg/l Chloride (Cl⁻) 320,000-370,000 mg/l Magnesium (Mg⁺²) 90,000-95,000 mg/l Potassium (K⁺) 1300-1500 mg/l Sodium (Na⁺) 1500-2500 mg/l Bromide (Br⁻) 11,000-12,000 mg/l Strontium (Sr⁻²) 750-850 mg/l.

The term “pharmaceutical composition” and ‘anti-inflammatory pharmaceutical composition’ relates interchangeably to an excipient phase such as an emulsion, gel or powder compatible for application on a skin of a human which is mixed with active ingredients and other additives. The preferable excipient phase relates to oil-in-water (o/w) or oil-in-water-in-oil (o/w/o) or water-in-oil-in-water (w/o/w) emulsions in the form of various viscosity grades including milk, lotion, cream and ointment and may include additives such as allantoin; SG or DPG, perfumes, preservatives, emulsifiers, active ingredients such as but not limited to: zinc oxide; plant extract (e.g., aloe barbadensis extract, hypophea etc), vitamins (e.g., tocopherol, ascorbic acid etc.), antioxidants and other ingredients that have an active therapeutic effect on pruritus and/or for easing the related symptoms of pruritus. It is yet in the scope of the present invention wherein the pharmaceutical composition relates to a gel or to a powder.

The term “Dead Sea minerals enriched pharmaceutical composition” relates to a mixture that is composed by the aforementioned pharmaceutical composition admixed with said Dead Sea minerals.

The term “nano-emulsion” refers to an emulsion in which the droplets or the particles in the dispersed phase have a diameter of less than 400 nanometers (0.4 microns) and usually less then 100 nm.

The term “surfactants” refers to chemical additives (non-ionic, cationic, anionic, Tween™ etc.) to the emulsion that stabilize the droplets by interacting with their surface, thus avoiding inter-surface interaction to form larger droplets.

In the preferred embodiment of this invention the Dead Sea minerals enriched pharmaceutical composition consists of: 1 to 6 wt. % Dead Sea Mud and optionally up to 4% Dead Sea water as active ingredients in a cosmetic emulsion (oil-in-water, water-in-oil, water-in-oil-in-water, or oil-in-water-in-oil) comprising of ingredients suitable for the preparation of a cosmetic cream such as ingredients selected from octyl palmitate, cetearyl alcohol (and) ceteareth-30, hexadecanol, glyceryl stearate, glycerin, PEG-40 stearate, Tween 60™, zinc oxide, zinc stearate, kaolin, and silica, propylene glycol, dipropylene glycol, aloe barbadensis extract, grape seed extract, green tea extract, hamamelis virginiana (witch hazel) distillate, hypophea oil, dimethicone, cyclomethicone, sorbitan tristearate, allontoin, methylparaben, propylparben, ascorbic acid, mineral salts of ascorbic acid, vitamin E, tocotrienol, retinyl palmitate, retinyl palmitate polypeptide, and di panthenol, glycine, lysine, selenomethionine, tyrosine, praline, coenzyme Q10, selenium, alpha lipoic acid, ascorbyl palmitate, and resveratrol, bronopol and fragrance.

In another embodiment of the present invention the excipient phase is a nano-emulsion as defined above. Preferably the nano-emulsion contains herbal oil droplets in the size of a few dozens of nanometers, preferably between 40 to 60 nanometers, which have an antiseptic potency as well as an increased ability to penetrate and absorb the active ingredients into the skin.

The preparation of the Dead Sea minerals enriched anti inflammatory pharmaceutical composition of the present invention is preferably done by several steps, e.g.:
1) heating distilled water to a temperature between 65° C. to 85° C., preferably about 75° C.;
2) adding water miscible ingredients to the aqueous phase until all ingredients are homogenously dissolved;
3) heating the selected oils in a separate reactor to a temperature between 65° C. to 85° C., preferably about 75° C.;
4) adding lipophilic ingredients to the oily phase until all ingredients are homogenously dissolved
5) dropwise introduction of the lipophilic phase into the reactor containing the aqueous phase while vigorously stirring and maintaining at a temperature between 65° C. to 85° C., preferably about 75° C.; or in other cases, dropwise introduction of the aqueous phase into the reactor containing the lipophilic phase while vigorously stirring and maintaining at a temperature between 55° C.
6) slowly cooling said mixture obtained in step 3 and adding heat sensitive ingredients at about 45° C.;
5) adding to the mixture said Dead Sea mud and Dead Sea water while stirring at about 25° C.; and,
6) removing the resultant emulsion from the reactor into suitable storage vessels.

The present invention further discloses a method of treating anti inflammatory conditions, especially, e.g., itching, dryness, peeling, tightness, participant cutaneous dryness and/or improving of sleep quality by applying a measurable of a Dead Sea salts or water-based emulsion as defined in any of the above.

An experiment that illustrates the effectiveness and safety of the pharmaceutical composition and the medical treatment involving the application of said pharmaceutical composition to patients carrying the described disorders is underway. Said experiment includes a single-center, randomized, double blind, double placebo-controlled clinical trial that studies the effects of topical application DS vs. P1 and DS vs. P2 (where DS is the Dead Sea enriched pharmaceutical composition, P1 is placebo lotion identical to DS minus Dead Sea minerals only and P2 is a placebo lotion with no active ingredients) in reducing symptoms of uremic pruritus (itching, dryness, peeling, tightness) in patients suffering from pruritis and especially in stable maintenance hemodialysis patients, that refrain from any antipruritic treatment, oral or topical, for a period of not less than two weeks prior to study initiation.

Inflammation in skin is characterized by distinct physiological responses the production of cytokines (as IL-1, IL-6, IL-7, IL-8, IL-10, IL-12, IL-15, TNF α), and the rise in reactive oxygen species (ROS). The latter play a pivotal role in the intracellular signal transduction pathways. Leading to stress and inflammation responses and, occasionally, to apoptotic death of cells.

Myers' Skin Biology and Biochemistry Laboratory of The Hebrew University is engaged in skin biochemistry research. Using skin organ culture model (ex-vivo), we have screened the capability of “anti-inflammatory” materials to regulate inflammation processes induced either by UVB irradiation (increased production and secretion of cytokines of TNF α and IL1) or by mechanical stress (surgery and tissue cutting increased production of IL 6 and IL 8). We found that the treatment of skin with the anti-inflammatory pharmaceutical composition of the present invention, also known as Dermud for hours prior to the UVB irradiation decreased the release of TNF α and IL 1 for almost 60 and 80% of control respectively at 24 hours after the irradiation.

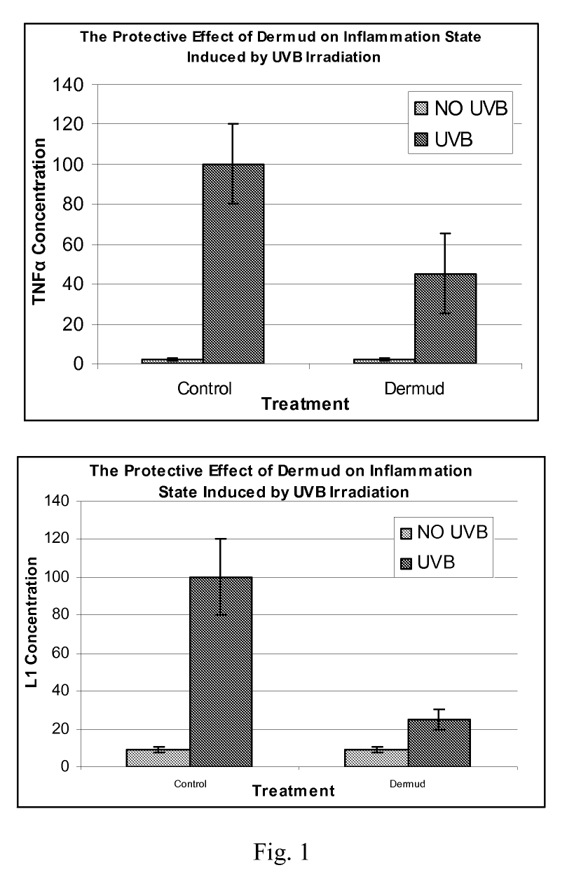

FIG. 1 discloses the protection effect of Dermud against inflammation induced by UVB Human skin pieces smeared with Dermud were incubated in DMEM medium for 36 hr at 37° C. 5% co2. samples were then UVB irradiated and re-incubated for the next 24 hours, when a sample medium was taken for cytokine determination (TNF α and IL1)

Determination of accumulated cytokines IL6 and IL8 released by the skin at 24 hours after UVB irradiation, (preceded by the topical application of Dermud for 36 hours), revealed a decrease of about 90% for IL6 and 80% for IL8 relative to the control

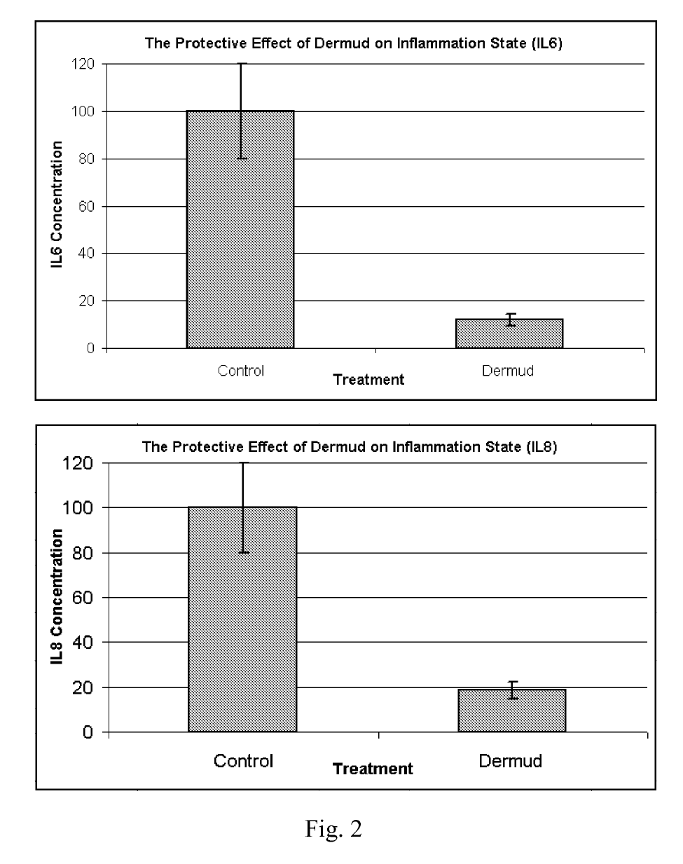

FIG. 2 discloses the effect of Dermud on reducing inflammatory cytokines Upon UVB irradiation UVB Human skin pieces smeared with Dermud were incubated in DMEM medium for 36 hr at 37° C. 5% co2. The medium was replaced with the fresh one and cultures re-incubated. After 24 hours samples of cell culture medium were taken for cytokine determination. The results indicate that Dermud could inhibit inflammatory response in skin induced by UVB irradiation or by mechanical stress.

What is claimed is:

1. An anti-inflammatory pharmaceutical composition, adapted to be applied to human skin and to be left on said skin for a time sufficient for said skin to substantially completely absorb at least an active ingredient in said pharmaceutical composition, wherein said pharmaceutical composition comprises said active ingredient comprising about 0.1 to 6 wt. % of a Dead Sea mud, preferably wt 0.15% to wt 0.20% suspension, and wherein the amount of said active ingredient is sufficient to ameliorate at least one disorder of said skin.

2. The anti-inflammatory pharmaceutical composition according to claim 1, wherein said pharmaceutical composition further comprises at least one member selected from the group consisting of fatty alcohols and emulsifiers, bulking agents, herbal extracts, vitamins and vitamin derivatives, amino acids, minerals, anti-oxidants, preservatives, and silicones.

3. The anti-inflammatory pharmaceutical composition according to claim 2, wherein the fatty alcohols and emulsifiers comprise one or more members selected from the group consisting of octyl palmitate, cetearyl alcohol, ceteareth-30, hexadecanol, glyceryl stearate, glycerin, PEG-40 stearate, propylene glycol, dipropylene glycol and sorbitan tristearate.

4. The anti-inflammatory pharmaceutical composition according to claim 2 wherein the bulking agents comprise one or more members selected from the group consisting of zinc oxide, zinc stearate, kaolin, calamin, silica and other minerals.

5. The anti-inflammatory pharmaceutical composition according to claim 2 wherein the herbal extracts comprise one or more members selected from the group consisting of aloe barbadensis extract, grape seed extract, green tea extract, hamamelis virginiana (witch hazel) distillate and hypophae oil.

6. The anti-inflammatory pharmaceutical composition according to claim 2 wherein vitamins and their derivatives comprise one or more members selected from the group consisting of ascorbic acid, mineral salts of ascorbic acid, vitamin E, tocotrienol, retinyl palmitate, retinyl palmitate polypeptide, and di panthenol.

7. The anti-inflammatory pharmaceutical composition according to claim 2 wherein the amino acids comprise one or more members selected from the group consisting of glycine, lysine, selenomethionine, tyrosine and proline.

8. The anti-inflammatory pharmaceutical composition according to claim 2 wherein the antioxidants comprise one or more members selected from the group consisting of coenzyme Q10, selenium, alpha lipoic acid, ascorbyl palmitate, and resveratrol.

9. The anti-inflammatory pharmaceutical composition according to claim 2 wherein the silicones comprise one or more members selected from the group consisting of dimethicone and cyclomethicone.

10. The anti-inflammatory pharmaceutical composition according to claim 2 wherein the emulsion is selected from the group consisting of an emulsion which further includes fragrance or essential oils; oil-in-water emulsion; water-in-oil-in-water emulsion; oil-in-water-in-oil emulsion; water-in-oil emulsion; an emulsion comprising non-ionic surfactants, anionic surfactants, cationic surfactants or a combination thereof;

11. The anti-inflammatory pharmaceutical composition according to claim 1 wherein either the droplets emulsified in the continuous phase or the solid particles suspended in the continuous phase have a diameter of less then 1,000 nanometers, preferably less than 200 nanometers.

12. An anti-inflammatory pharmaceutical composition according to claim 1, wherein the emulsion is especially adapted to treating animals.

13. A method for the preparation of an anti-inflammatory pharmaceutical composition that is adapted to treat cutaneous dryness, itching, peeling and tightness, especially in hemodialysis patients in a human and is adapted to moisturize said skin, comprising: a) preparing an oil-in-water emulsion with at least one non-ionic emulsifier(s); and b) adding about 0.1-10 wt. % of a Dead Sea mud suspension to said emulsion.

14. The method according to claim 13, further comprising step of adapting the anti-inflammatory pharmaceutical composition to treat an animal.

15. The method according to claim 13 wherein the at least one of said non-toxic emulsifiers is selected from the group consisting of ethoxilated sorbitan esters, sorbitan esters, ethoxilated alcohols, or fatty acids, alkyl glycosides, and mono- and di-glycerides.

16. A method for treating at least one skin condition that comprises steps of topically administrating of a pharmaceutical composition, comprising about 0.1 to 6 wt % of a suspension of active ingredient comprising a Dead Sea mud suspension, to skin suffering from a disorder; and leaving said pharmaceutical composition on said skin until at least substantially all of said Dead Sea water has been absorbed by the skin and said disorder has been ameliorated.

17. The method described in claim 16 comprising repeating the treatment several times as long as needed, preferably 1 to 4 times a day for 1 to 30 consecutive days, ideally twice a day for 14 consecutive days, preferably after rinsing the skin.

18. The method as claimed in claim 16 comprising a step of providing the pharmaceutical emulsion with at least about 4 wt % of Dead Sea water.

19. The method as claimed in claim 16 comprising a step of leaving the pharmaceutical emulsion on the skin until at least substantially all of said Dead Sea water has been absorbed by the skin and said disorder has been ameliorated.

20. The method as claimed in claim 16, comprising a step of adapting the pharmaceutical emulsion to an animal skin.

21. The anti-inflammatory pharmaceutical composition as claimed in claim 1 wherein the Dead Sea mud is replaced by a similar composition that does not originate from the Dead Sea.

22. The anti-inflammatory pharmaceutical composition as claimed in claim 1 wherein the Dead Sea water is replaced by a similar solution of minerals that do not originate from the Dead Sea.

23. The anti-inflammatory pharmaceutical composition as claimed in claim 1 wherein the excipient phase is a powder.

24. The anti-inflammatory pharmaceutical composition as claimed in claim 1, wherein the excipient phase is a gel.
